(19) United States
(12) Patent Application Publication
(10) Pub. No.: US 2024/0189311 A1
(43) Pub. Date: Jun. 13, 2024

(54) Combination Therapy of Obicetrapib and Ezetimibe for Use in Statin Intolerant Patients Suffering from Hyperlipidemia or Mixed Dyslipidaemia

(71) Applicant: NEWAMSTERDAM PHARMA B.V., Naarden (NL)

(72) Inventors: Johannes Jacob Pieter Kastelein, Blaricum (NL); Marc Ditmarsch, Den Haag (NL); Michael Harvey Davidson, Sunny Isles, FL (US)

(21) Appl. No.: 18/277,851

(22) PCT Filed: Feb. 18, 2022
(86) PCT No.: PCT/NL2022/050083

§ 371 Date: Aug. 18, 2023

(30) Foreign Application Priority Data
Feb. 18, 2021 (WO) PCT/NL2021/050109

(51) Int. Cl. A61K 31/506; A61K 31/397; A61P 3/06

(52) U.S. Cl. CPC A61K 31/506; A61K 31/397; A61P 3/06

(57) ABSTRACT

The present invention relates to a pharmaceutical composition and a therapeutic combination comprising obicetrapib and ezetimibe, which may be used in the treatment of subjects suffering from or having an increased risk for hyperlipidemia or mixed dyslipidemia and wherein said subjects are partially or completely intolerant to statins.

### TECHNICAL FIELD OF THE INVENTION

The present invention relates to a pharmaceutical composition and a therapeutic combination comprising obicetrapib and ezetimibe for use in the treatment of partially or completely statin intolerant subjects suffering from hyperlipidemia or mixed dyslipidemia or subjects having an increased risk for hyperlipidemia or mixed dyslipidemia.

### BACKGROUND OF THE INVENTION

Despite advances in treatment, cardiovascular disease (CVD) is the leading cause of death globally, resulting in over 17 million deaths annually. For many years it has been known that abnormal cholesterol levels have been associated with increased risk of cardiovascular disease (CVD), such as cardiomyopathy, atherosclerosis and myocardial infarction. In particular, individuals presenting with high levels of low-density lipoprotein (LDL) cholesterol and very-low-density lipoprotein (VLDL) cholesterol combined with low levels of high-density lipoprotein (HDL) cholesterol were observed to be at the highest risk of developing a cardiovascular disease.

3-Hydroxy-3-methylglutaryl coenzyme A reductase (HMG-CoA reductase) inhibitors, more commonly known as statins, were found to be capable of lowering the level of LDL cholesterol in the blood of patients by reducing the production of cholesterol and accelerating cell uptake of cholesterol from the bloodstream. The use of statins to combat high levels of cholesterol, also known as hypercholesterolemia, proved to be successful as the LDL cholesterol levels were decreased to such a degree that a lower risk of developing cardiovascular disease was observed.

However, there are many safety issues associated with the use of statins. For instance, statins may lead to increases in liver enzymes and myopathy, rhabdomyolysis, which can lead to acute renal failure, and unexplained muscle pain and weakness. These manifestations, and other adverse events (AEs), may lead to discontinuation of statin therapy or the use of low doses of statins, which are less frequently associated with side effects. This is commonly referred to as statin intolerance (or statin toxicity) and may occur in a considerable percentage of patients, depriving such patients of the full cardioprotective benefits of statins. In addition, discontinuation of statin therapy can increase the risk of acute cardiovascular events.

Another class of compounds having anti-hyperlipidemic effects are the so-called cholesterol absorption inhibitors (CAIs). CAIS are capable of preventing the uptake of cholesterol from the small intestine by blocking the uptake of micellar cholesterol, which reduces the incorporation of cholesterol esters into chylomicrons and chylomicron remnants. CAIs thereby reduce the amount of cholesterol that is circulated back to the liver, which in turn increases the activity of hepatic LDL-receptors and increases the clearance of LDL cholesterol particles from the bloodstream.

A known example of a CAI is ezetimibe, previously known as compound “Sch-58235” of Schering-Plough, and marketed amongst others under the brand names Ezetrol and Zetia (Merck Sharp & Dohme/Merck). The IUPAC name of ezetimibe is (3R,4S)-1-(4-fluorophenyl)-3-[(3 S)-3-(4-fluorophenyl)-3-hydroxypropyl]-4-(4-hydroxyphenyl)azetidin-2-one.

Ezetimibe is administered frequently either as a mono-therapy, or in an add-on combination therapy approach together with one of several statins. Ezetimibe selectively inhibits dietary and biliary cholesterol absorption by binding to the protein encoded by the Niemann-Pick C1-like 1 gene at the brush border membrane of enterocytes. Typically, the ezetimibe dosage form is a tablet comprising 10 mg ezetimibe, for oral administration. Ezetimibe as a mono-therapy has been shown to moderately reduce LDL-C levels in patients, i.e. a reduction of less than 20% when compared to placebo, in patients with hypercholesterolemia.

Another approach is to inhibit the Cholesteryl Ester Transfer Protein (CETP). CETP is a plasma protein secreted primarily by liver and adipose tissue. CETP mediates the transfer of cholesteryl esters from HDL to apolipoprotein B (ApoB)-containing particles (mainly LDL and VLDL) in exchange for triglycerides (TG), thereby decreasing the cholesterol content in HDL in favor of that in (V)LDL. Hence, CETP inhibition has been hypothesized to retain cholesteryl esters in HDL-C and decrease the cholesterol content of the atherogenic ApoB fraction.

Despite the evidence supporting the potential of CETP inhibition in reducing cardiovascular morbidity, clinical development of CETP inhibitors has not been straightforward. The first compound to progress to Phase III clinical trials was torcetrapib which was dosed at 60 mg. Torcetrapib was shown to increase HDL-C by 72% and decrease LDL-C by 25%, but it was subsequently withdrawn from development owing to safety concerns including an unexpected increase in cardiovascular events and death.

Although the mechanism of those events is not fully understood, there is increasing evidence that they might have been due to off-target effects of torcetrapib such as increased blood pressure, changes in electrolytes (increases in sodium and bicarbonate and decreases in potassium) and increases in aldosterone, consistent with mineralocorticoid activity.

Subsequently, another CETP inhibitor, dalcetrapib, entered clinical trials. Dalcetrapib was shown to be a weak inhibitor that increased HDL-C by 30-40% with minimal effects on LDL-C concentrations.

Two more CETP inhibitors, anacetrapib (MK-0859 of Merck Sharp & Dohme Corp.) and evacetrapib, were also tested in Phase III clinical trials. Evacetrapib failed to pass a futility interim analysis, but anacetrapib showed in the 30.000 patient REVEAL trial that it reduced major cardiovascular events (MACE) through its lowering of LDL-cholesterol. However, the development of both compounds has been terminated.

This leaves obicetrapib (also known as TA-8995) as the only CETP inhibitor currently under full development. In the Phase II trial (TULIP; Hoving et al., 2015) it was found that both 10 mg and 5 mg obicetrapib administered as monotherapy was able to reduce LDL cholesterol concentrations. It was also found that in combination with 20 mg atorvastatin the total reduction of LDL cholesterol was even higher. Moreover, both in case of mono-therapy and in combination with statins the HDL cholesterol concentration increased considerably.

However, not all patients tolerate statins well. Hence, a continuing need remains for convenient, safe and effective agents or combination of agents for use in the treatment of subjects suffering from or having an increased risk for hyperlipidemia or mixed dyslipidemia and to the reduce the risk for cardiovascular events.

### SUMMARY OF THE INVENTION

A first aspect of the present invention relates to a pharmaceutical composition comprising obicetrapib and ezetimibe or pharmaceutically acceptable salts or solvates thereof and a pharmaceutically acceptable carrier for use in the treatment of subjects suffering or having an increased risk for hyperlipidemia or mixed dyslipidemia and wherein said subjects are partially or completely intolerant to statins.

A second aspect of the present invention relates to a therapeutic combination comprising obicetrapib and ezetimibe or pharmaceutically acceptable salts or solvates thereof for use in the treatment of subjects suffering from hyperlipidemia or mixed dyslipidemia or having an increased risk for hyperlipidemia or mixed dyslipidemia and wherein said subjects are partially or completely intolerant to statins.

It has surprisingly been found that with said composition and combination it is possible to reduce both the LDL-C and ApoB concentration. It has even more surprisingly been found that with said combination of obicetrapib and ezetimibe the ApoB concentration can be reduced considerably more than was to be expected on the basis of previous experiences with the combination of CETP-inhibitors and statins in general, and obicetrapib with statins specifically. This makes the present invention particularly relevant for partially or completely statin intolerant patients.

Obicetrapib, also referred to as “TA-8995”, has the following chemical structure:

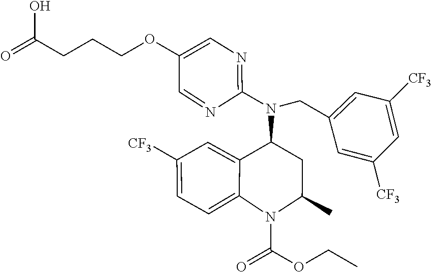 [SMILES: CCOC(=O)N1c2ccc(C(F)(F)F)cc2[C@@H](N(Cc2cc(C(F)(F)F)cc(C(F)(F)F)c2)c2ncc(OCCCC(=O)O)cn2)C[C@H]1C]

Ezetimibe, also referred to as “Sch-58235”, has the following chemical structure:

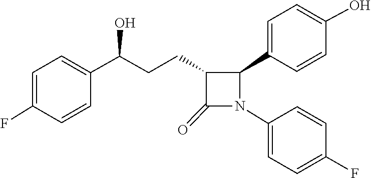 [SMILES: O=C1[C@H](CC[C@H](O)c2ccc(F)cc2)[C@@H](c2ccc(O)cc2)N1c1ccc(F)cc1]

Within the present invention both obicetrapib and ezetimibe may also be used as different salt forms and solvates. They may also be formulated as pro-drugs.

### DEFINITIONS

The term “apolipoprotein” as used herein has its conventional meaning and refers to proteins that bind lipids to form lipoproteins.

The term “apolipoprotein B” (ApoB) as used herein has its conventional meaning and refers to the protein encoded by the ApoB gene.

The term ‘pharmaceutical composition’ as used herein has its conventional meaning and refers to a composition which is pharmaceutically acceptable.

The term ‘pharmaceutically acceptable’ as used herein has its conventional meaning and refers to compounds, material, compositions and/or dosage forms, which are, within the scope of sound medical judgment suitable for contact with the tissues of mammals, especially humans, without excessive toxicity, irritation, allergic response and other problem complications commensurate with a reasonable benefit/risk ratio.

The term “carrier” as used herein has its conventional meaning and refers to a pharmaceutically acceptable diluent, adjuvant, excipient or vehicle with which a pharmaceutically active ingredient is administered.

The term ‘excipient’ as used herein has its conventional meaning and refers to a pharmaceutically acceptable ingredient, which is commonly used in the pharmaceutical technology for preparing a granulate, solid or liquid oral dosage formulation.

The term salt' as used herein has its conventional meaning and includes the acid addition and base salts of a pharmaceutically active compound.

The term “solvate” as used herein has its conventional meaning and refers to a compound formed by solvation, for example as a combination of solvent molecules with molecules or ions of a solute. Well known solvent molecules include water, alcohols, nitriles and polar organic solvents.

The term “subject” as used herein refers to humans suffering from or at risk for a certain disease or disorder. The term “subject” and “patient” herein are used interchangeably.

The term ‘increased risk’ has its conventional meaning and refers to a situation in a subject, preferably human, where in individuals, either male or female, have an LDL-cholesterol level above 2.6 mmol/l (100,54 mg/dL), such that they are exposed at an increased risk of a cardiovascular event, compared to those with lower levels.

The term ‘treatment’ as used herein has its conventional meaning and refers to curative, palliative and prophylactic treatment.

The term ‘cardiovascular disease’ as used herein has its conventional meaning and includes clinical manifestations of arteriosclerosis, peripheral vascular disease angina, ischemia, cardiac ischemia, stroke, myocardial infarction, reperfusion injury, restenosis after angioplasty, hypertension, cerebral infarction and cerebral stroke.

The term “cardiovascular event” as used herein has its conventional meaning and refers to occurrence of myocardial infarction, coronary revasculization, stroke, or coronary death (Ference, 2017).

The term “hypercholesterolemia” as used herein has its conventional meaning and refers to the condition in which high levels of cholesterol are present in the blood.

The term “hyperlipidaemia” as used herein has its conventional meaning and refers to the condition in which there are high amounts of lipids found in the blood.

The term “mixed dyslipidaemia” as used herein has its conventional meaning and refers to the condition in which there are elevations of LDL cholesterol and triglyceride levels that are accompanied by low levels of HDL cholesterol in the blood.

The term “statin intolerant” as used herein has its conventional meaning and refers to subjects inability to tolerate two or more statins, one at a low dose, due to an adverse safety effect that started or increased during statin therapy and resolved or improved when statin was discontinued, reference is in this regard also made to the similar definition approved by the FDA in the bempedoic acid (Esperion) phase III trial.

The term ‘cholesterol absorption inhibitor’ (CAI) as used herein has its conventional meaning and refers to compounds which are used to lower LDL-C by blocking enteric and biliary absorption of cholesterol. A known cholesterol absorption inhibitor is ezetimibe.

The term ‘HMG-CoA reductase inhibitor’ as used herein has its conventional meaning and is used interchangeably with the term ‘statins’ and refers to compounds which are used to lower LDL-C by inhibiting the enzyme HMG-CoA reductase. Well known HMG-CoA reductase inhibitors are atorvastatin, pravastatin, fluvastatin, simvastatin, lovastatin, rosuvastatin and pitavastatin.

The term “cholesteryl ester transfer protein inhibitor” (CETP inhibitor) as used herein has its conventional meaning and refers to a class of compounds that inhibits the CETP receptor in mammals. A known CETP inhibitor is obicetrapib.

The term ‘unit dosage form’ has its conventional meaning and refers to a dosage form which has the capacity of being administered to a subject, preferably a human, to be effective, and which can be readily handled and packaged, remaining as a physically and chemically stable unit dose comprising the therapeutic agent, i.e. obicetrapib or combination of therapeutic agents, such as obicetrapib and ezetimibe.

The term ‘fixed dose combination’ as used herein has its conventional meaning and refers to a combination of defined doses of two or more drugs or active ingredients presented in a single dosage unit (e.g. a tablet or a capsule) and administered as such.

The term ‘free dose combination’ as used herein has its conventional meaning and refers to a combination of two drugs or active ingredients administered simultaneously but as two distinct dosage units.

### DETAILED DESCRIPTION OF THE INVENTION

A first aspect of the present invention relates to a pharmaceutical composition comprising obicetrapib and ezetimibe or pharmaceutically acceptable salts or solvates thereof and a pharmaceutically acceptable carrier for use in the treatment of subjects suffering or having an increased risk for hyperlipidemia or mixed dyslipidemia and wherein said subjects are partially or completely intolerant to statins.

A second aspect of the present invention relates to a therapeutic combination comprising obicetrapib and ezetimibe or pharmaceutically acceptable salts or solvates thereof for use in the treatment of subjects suffering from hyperlipidemia or mixed dyslipidemia or having an increased risk for hyperlipidemia or mixed dyslipidemia and wherein said subjects are partially or completely intolerant to statins.

It has surprisingly been found that the reduction of the LDL concentration in said patients was considerably more than was to be expected on the basis of previous experiences with the combination of CETP-inhibitors and statins. In fact it has surprisingly been found that the combined use of obicetrapib and ezetimibe has a strong synergistic effect in patients treated with said combination. This makes the present invention particularly relevant for partially or completely statin intolerant patients.

The inventors have found that the use of ezetimibe did not attenuate the LDL-C lowering efficacy of obicetrapib hic remained the same as with obicetrapib monotherapy. In other words, the relative LDL-C reduction by obicetrapib when administered in combination with ezetimibe remained similar to the LDL-C reduction by the same dose obicetrapib administered as monotherapy.

This improved LDL-C lowering efficacy is important in view of the fact that the causal effect of CETP inhibition on the risk of cardiovascular events is to be determined by changes in the concentration of LDL-C levels (Ference et al., 2017).

A Mendelian randomization analysis (unpublished) revealed that the previously observed CETP inhibitor and HMGCR inhibitor interaction (i.e. attenuation of the effect of CETP inhibition when combined with HMGCR inhibitors) does not occur when a CETP inhibitor is combined with NPC1L1 inhibitors, such as ezetimibe. From this analysis it is further clear that the LDL-C lowering efficacy of CETP inhibitors in general (and obicetrapib specifically) is not affected (i.e. attenuated) when used in combination with ezetimibe. The results of this analysis are provided in the experimental part below.

Moreover, these results show that by using a combination of obicetrapib and ezetimibe in said patients the LDL-C lowering efficacy of obicetrapib is not attenuated and that such use will result in an increased LDL-C lowering. In view of the fact the causal effect of CETP inhibition on the risk of cardiovascular events is to be determined by the changes in the concentration of LDL-C it is also shown that with the pharmaceutical composition and therapeutic combination according to the present invention the risk for cardiovascular events is reduced.

Hence, the pharmaceutical composition or therapeutic combination according to the present invention is preferably used to reduce the risk for cardiovascular events in patients suffering or having an increased risk for hyperlipidemia or mixed dyslipidemia.

The pharmaceutical composition or therapeutic combination according to the present invention may also be used for the treatment of mild dyslipidemia.

Furthermore, a subject in need thereof is preferably administered by means of said pharmaceutical composition or therapeutic combination between 1 to 10 mg per day obicetrapib and between 5 to 20 mg ezetimibe per day. More preferably, said subject is administered about 5 mg obicetrapib per day and about 10 mg ezetimibe per day or about 10 mg obicetrapib per day and about 10 mg ezetimibe per day.

The pharmaceutical composition or therapeutic combination according to the present invention may thus be used for reducing the LDL-C concentration in the blood of said subjects. As described in Ference et al., 2017 it is particularly such an LDL-C lowering effect that reduces the risk for cardiovascular events.

The LDL-C concentration in the blood of said subjects is when administered a combination ezetimibe and obicetrapib preferably about 50% less when compared to placebo and about 40% when compared to ezetimibe monotherapy.

In other words, with the pharmaceutical composition or the therapeutic combination according to the present invention it is has become possible to maintain the relative LDL-C reduction by obicetrapib when administered in combination with ezetimibe similar to the relative LDL-C reduction by the same dose of obicetrapib administered as monotherapy, i.e. with the combination of the present invention the CETP inhibitory effect of obicetrapib is not attenuated

More specifically, the relative LDL-C reduction by a daily dose 5 mg obicetrapib when administered in combination with 10 mg ezetimibe remains similar to the relative LDL-C reduction by a daily dose of 5 mg obicetrapib administered as monotherapy. Moreover, a similar effect on relative LDL-C reduction is observed when a combined dose of 10 mg obicetrapib per day and 10 mg ezetimibe per day is administered and compared to 10 mg obicetrapib monotherapy.

The pharmaceutical composition or therapeutic combination according to the present invention is preferably administered orally to subjects in need thereof. Oral administration may involve swallowing, so that the pharmaceutically active compounds enter the gastrointestinal tract. Specific pharmaceutical preparations, as described below, may be developed which facilitate the oral administration.

The pharmaceutical composition according to the present invention is preferably formulated as an oral free dose combination or as an oral fixed dose combination, more preferably as an oral fixed dose combination. The different pharmaceutically active ingredients may be present in said combinations as granulates. Preferably, the pharmaceutical composition is an oral fixed dose combination, such a combination is very convenient for patients and avoids problems with administering the correct amounts of these compounds.

Solid oral dosage forms which may be used within the context of the present invention include besides tablets and capsules amongst others caplets, lozenges, pills, mini-tablets, pellets, beads and granules. Liquid oral dosage forms which may be used for the pharmaceutical preparation of the present invention include, but are not limited to drinks, solutions, suspensions, syrups, beverages and emulsions.

The oral fixed dose combination or oral free dose combination is preferably formulated as a solid dosage form, such as a tablet or capsule. Generally, the administration of these kinds of formulations is considered to be the most convenient for patients.

In a preferred embodiment the pharmaceutical composition or therapeutic combination according to the present invention is an oral fixed dose combination comprising about 1 to about 10 mg obicetrapib and about 5 to about 20 mg ezetimibe, more preferably said composition or combination comprises about 5 mg obicetrapib and about 10 mg ezetimibe or about 10 mg obicetrapib per day and about 10 mg ezetimibe per day.

Besides obicetrapib and ezetimibe, pharmaceutically acceptable salts thereof may also be used in the pharmaceutical composition or therapeutic combination according to the present invention. Pharmaceutically acceptable salts of obicetrapib and ezetimibe include the acid addition and base salts thereof, such as preferably the calcium, potassium or sodium salts. For a review on suitable salts, reference is made “Handbook of Pharmaceutical Salts: Properties, Selection, and Use” by Stahl and Wermuth (Wiley-VCH, Weinheim, Germany, 2002).

A pharmaceutically acceptable salt of obicetrapib or ezetimibe may be readily prepared by mixing together solutions of such compounds and the desired acid or base, as appropriate. The salt may precipitate from solution and be collected by filtration or may be recovered by evaporation of the solvent.

The present invention also relates to the use of pharmaceutically acceptable solvates or pro-drugs of obicetrapib and/or pharmaceutically acceptable solvates or pro-drugs of ezetimibe, in the pharmaceutical composition or therapeutic combination of the present invention.

In an embodiment of the present invention, the composition or combination according the present invention also comprises polyunsaturated fatty acids (PUFAs), preferably omega-3 polyunsaturated fatty acids, more preferably PUFAs chosen from the group consisting of eicosapentaenoic acid (EPA), docosahexaenoic acid (DHA), docosapentaenoic acid (DPA) or combinations thereof. Preferably, PUFAs are present in their free acid form, in the pharmaceutical composition or combination according to the present invention.

PUFA's, in particular omega-3 PUFAs, have a specific capacity against triglyceride rich lipoproteins, remnant cholesterol and small dense LDL, whereas CETP-inhibitors have no or little effect against triglyceride rich lipoprotein and remnant cholesterol. Hence, combining obicetrapib, ezetimibe and PUFAs in a pharmaceutical composition makes such a composition particularly suitable for the treatment of subjects suffering from or having an increased risk for cardiovascular diseases.

In this regard it is also noted that due to the relatively low amounts of obicetrapib and ezetimibe which are needed to obtain a clinically relevant effect, it is possible that oral formulations of the composition according to the present invention comprise relatively high amounts of PUFA's (e.g. per capsule).

The pharmaceutical composition or therapeutic combination according to the present invention comprises besides obicetrapib and ezetimibe also a pharmaceutically acceptable carrier and excipient. Examples of categories of excipients include, but are not limited to, binders, disintegrants, lubricants, glidants, fillers and diluents. One of ordinary skill in the art may select one or more of the aforementioned excipients with respect to the particular desired properties of the granulate and/or solid oral dosage form by routine experimentation and without any undue burden. The amount of each excipient used may vary within ranges conventional in the art. The following references which are all hereby incorporated by reference disclose techniques and excipients used to formulate oral dosage forms. See “The Handbook of Pharmaceutical Excipients”, 4th edition, Rowe et al., Eds., American Pharmaceuticals Association (2003); and “Remington: The Science and Practice of Pharmacy”, 20th edition, Gennaro, Ed., Lippincott Williams & Wilkins (2000).

A further aspect of the present invention relates to method of treating a partially or completely statin intolerant subject suffering from or having an increased risk for hyperlipidemia or mixed dyslipidemia, the method comprising administering to said subject in need thereof an effective dosage amount of the pharmaceutical composition or therapeutic combination according to any of the previous claims.

As explained in more detail below, said method may be used for reducing the risk for cardiovascular events.

A further aspect of the present invention relates to a pharmaceutical composition or therapeutic combination in the manufacture of a medicament for use in the treatment of partially or completely statin intolerant subjects suffering from or having an increased risk for hyperlipidemia or mixed dyslipidemia.

A last aspect of the present invention relates to a pharmaceutical composition or therapeutic combination comprising a CETP-inhibitor and ezetimibe for use in the treatment of partially or completely statin intolerant subjects suffering from cardiovascular diseases or having an increased risk for cardiovascular diseases. It is particularly used for reducing the risk for cardiovascular events. Furthermore, with said composition or combination the LDL-C concentration in the blood of said subjects may be reduced. Moreover, the relative LDL-C reduction by obicetrapib when administered in combination with ezetimibe is in a further embodiment similar to the relative LDL-C reduction by the same dose of obicetrapib administered as monotherapy.

The present invention will be illustrated further by means of the following non-limiting examples.

### EXAMPLES

### Example 1

A Mendelian randomization analysis was carried of 470.471 participants with individual level data from UK Biobank and dbGAP. From these participants 44.321 suffered from a major cardiovascular event (CHD death, MI, stroke or coronary revascularization). After analysis it was found that CETP inhibition is causally associated with decreased risk of major cardiovascular events. It was further found that a substantial attenuation of effect (i.e. the reduction of ApoB/LDL-C lowering) occurred when CETP inhibition is added to HMGCR inhibition. However, it was surprisingly found that a similar attenuation of CETP effect did not occur when added to NPC1L1 (i.e. CETP stratified by NPC1L1 inhibition). Hence, other than would have been expected on the basis of HMGCR inhibition by statins, the use of background ezetimibe does not lead to a attenuation in effect of CETP on lowering ApoB/LDL-C and thereby on cardiovascular events. This makes said combination very suitable for lowing the ApoB/LDL-C concentration in statin intolerant patients.

The results of the analysis are depicted below, wherein tertile 1 represents the lowest degree of inhibition of either HMGCR or NPC1LI and tertile 3 represents the highest degree of inhibition of said receptors. From these results it is clear that the more inhibition of HMGCR takes place, the smaller the effect of CETP inhibition on APO-B/LDL-C becomes. Remarkably, increased inhibition of NPC1L1 does not lead to a smaller effect of CETP inhibition on APO-B/LDL-C:

### Example 2a

Placebo-Controlled, Double-Blind, Randomized Phase 2 Study to Evaluate the Effect of Obicetrapib in Combination with Ezetimibe in Participants with Mild Dyslipidaemia and Potential Statin Intolerance (Protocol)

### Study Objectives

### Primary Objective

The primary objective of this study is to evaluate the effect of obicetrapib in combination with ezetimibe compared to placebo on low-density lipoprotein cholesterol (LDL-C) at Day 57.

### Secondary Objectives

The secondary objectives of this study include the following:
To evaluate the effect of obicetrapib monotherapy compared to placebo on LDL-C at Day 57;
To evaluate the effect of obicetrapib in combination with ezetimibe compared to placebo on apolipoprotein B (ApoB) at Day 57;
To evaluate the effect of obicetrapib monotherapy compared to placebo on ApoB at Day 57;
To evaluate the effect of obicetrapib in combination with ezetimibe compared to ezetimibe monotherapy on LDL-C at Day 57;
To evaluate the effect of obicetrapib monotherapy compared to ezetimibe monotherapy on LDL-C at Day 57;
To evaluate the effect of ezetimibe monotherapy compared to placebo on LDL-C at Day 57; and
To evaluate the effect of obicetrapib in combination with ezetimibe compared to ezetimibe monotherapy on ApoB at Day 57.
To evaluate the effect of said treatment on patients that are partly or completely statin intolerant as determined according to the FDA definition also used in the bempedoic acid (Esperion) phase III trial.

### Exploratory Objectives

The exploratory objectives of this study include the following:
To evaluate the effect of ezetimibe monotherapy compared to placebo on ApoB at Day 57;
To evaluate the effect of obicetrapib alone, ezetimibe alone, and obicetrapib in combination with ezetimibe on non-high-density lipoprotein cholesterol (non-HDL-C), very low-density lipoprotein cholesterol (VLDL-C), high-density lipoprotein cholesterol (HDL-C), and triglycerides (TG), apolipoprotein E (ApoE), and high-density lipoprotein (HDL)-ApoE (with and without apolipoprotein C3 [ApoC3]) at Day 57;
To evaluate the effect of obicetrapib alone, ezetimibe alone, and obicetrapib in combination with ezetimibe on the proportion of participants achieving predefined LDL-C targets at Day 57;
To assess the mean trough plasma levels of obicetrapib alone, ezetimibe alone, and obicetrapib in combination with ezetimibe at steady state on Day 57, Week 12, and Week 16;
To evaluate the effect of obicetrapib alone, ezetimibe alone, and obicetrapib in combination with ezetimibe on cholesteryl ester transfer protein (CETP) mass at Day 57, Week 12, and Week 16; and
To evaluate the safety and tolerability profiles of obicetrapib alone, ezetimibe alone, and obicetrapib in combination with ezetimibe assessed by clinical laboratory values and incidence of adverse events (AEs).

### Study Population

The population for this study includes men and women of 18 to 75 years of age, inclusive, with a body mass index of <40 kg/m2 and mild dyslipidaemia defined as fasting LDL-C levels of >2.5 mmol/L and <4.5 mmol/L and TG levels of <4.5 mmol/L.

### Sample Size Determination

A sample size of at least 100 evaluable participants (ie, 25 participants per treatment group) will provide >90% power to detect a 30% difference in LDL-C reduction at Day 57 (SD 15%) for the combination therapy group compared to the placebo group at a 2-sided significance level of 0.05.

The sample size for this study was determined in order to provide sufficient power (>80%) for the analyses of the primary efficacy endpoint and secondary efficacy endpoints described above. This sample size will also contribute sufficient participant exposure and safety data.

Participants will be stratified according to their Screening Visit (Visit 1) LDL-C value (<3.5 mmol/L or >3.5 mmol/L).

### Inclusion Criteria

Participants who meet all of the following criteria will be eligible to participate in the study:
1. Understanding of the study procedures, willingness to adhere to the study schedules and diet, and agreement to participate in the study by giving written informed consent prior to Screening procedures;
2. Men or women 18 to 75 years of age, inclusive;
   Women may be enrolled if all 3 of the following criteria are met:
      They are not pregnant;
      They are not breastfeeding; and
      They do not plan on becoming pregnant during the study;
   Women of childbearing potential must have a negative urine pregnancy test at the Screening Visit. Note: Women are not considered to be of childbearing potential if they meet 1 of the following criteria as documented by the Investigator:
      They have had a hysterectomy or tubal ligation at a minimum of 1 cycle prior to signing the ICF; or
      They are post-menopausal, defined as ≥1 year since their last menstrual period for women >55 years of age or >1 year since their last menstrual period and have a follicle-stimulating hormone (FSH) level in the menopausal range for women <55 years of age;
   Women of childbearing potential must agree to use an effective method of avoiding pregnancy from the Screening Visit to 90 days after the last visit. Men whose partners are of childbearing potential must agree to use an effective method of avoiding pregnancy from the Screening Visit to 90 days after the last visit. Effective methods of avoiding pregnancy are contraceptive methods with a Pearl index of <1 used consistently and correctly (including implantable contraceptives, injectable contraceptives, oral contraceptives, transdermal contraceptives, intrauterine devices, diaphragm with spermicide, male or female condoms with spermicide, or cervical cap) or a sterile sexual partner;
3. Fasting LDL-C levels >2.5 mmol/L and <4.5 mmol/L and TG levels <4.5 mmol/L (Visit 1); and
4. Willingness to maintain a stable diet and physical activity level throughout the study.

### Exclusion Criteria

Participants who meet any of the following criteria will be excluded from participation in the study:
1. Body mass index≥40 kg/m2;
2. Participation in another clinical study involving an investigational or marketed drug within 30 days prior to the Screening Visit;
3. Currently taking any lipid-altering therapy;
4. Any clinical manifestation of atherosclerotic CVD or any evidence of ischemic coronary disease present on the 12-lead ECG at the Screening Visit;
5. Diagnosis of type 1 or type 2 diabetes mellitus; or glycosylated hemoglobin (HbA1c)≥6.5% at the Screening Visit if no prior diagnosis of diabetes mellitus;
6. Uncontrolled hypertension, ie, sitting systolic blood pressure>160 mmHg and/or sitting diastolic blood pressure>90 mmHg. One retest will be allowed, at which point if the retest result is no longer exclusionary, the participant may be randomized;
7. Active muscle disease or persistent creatine kinase concentration>3×the upper limit of normal (ULN). One retest will be allowed after 1 week to verify the result, at which point if the retest result is no longer exclusionary, the participant may be randomized;
8. History of torsades de pointes;
9. Estimated glomerular filtration rate<60 mL/min calculated using the Chronic Kidney Disease Epidemiology Collaboration equation;
10. Hepatic dysfunction as evidenced by any laboratory abnormality as follows: gamma-glutamyl transferase, alanine aminotransferase, or aspartate aminotransferase>2×ULN, or total bilirubin>1.5×ULN;
11. Anaemia, defined as haemoglobin concentration<11 g/dL for males and haemoglobin concentration<9 g/dL for females;
12. History of malignancy within the past 5 years, with the exception of non-melanoma skin cancers;
13. Evidence of any other clinically significant non-cardiac disease or condition that, in the opinion of the Investigator, would preclude participation in the study; or
14. Known ezetimibe or CETP inhibitor allergy or intolerance.

### Withdrawal Criteria

Participation in this clinical study may be discontinued for any of the following reasons:
The participant withdraws consent or requests discontinuation from the study for any reason;
Occurrence of any medical condition or circumstance that exposes the participant to substantial risk and/or does not allow the participant to adhere to the requirements of the protocol;
Any serious adverse event (SAE), clinically significant AE, severe laboratory abnormality, intercurrent illness, or other medical condition which indicates to the Investigator that continued participation is not in the best interest of the participant;
Pregnancy;
Requirement of prohibited concomitant medication;
Participant failure to comply with protocol requirements or study-related procedures; or
Termination of the study by the Sponsor or the regulatory authority.

If a participant withdraws prematurely from the study due to the above criteria or any other reason, study staff should make every effort to complete the full panel of assessments scheduled for the Early Termination Visit. The reason for participant withdrawal must be documented in the electronic case report form (eCRF).

In the case of participants lost to follow-up, at least 3 attempts to contact the participant must be made and documented in the participant's medical records.

Withdrawn participants will not be replaced.

### Dosage Form and Route of Administration

The study drugs used in this study are as follows:
5 mg obicetrapib tablet;
10 mg ezetimibe tablet, over-encapsulated into a capsule;
Placebo tablet for obicetrapib; and
Placebo capsule for ezetimibe.

All products are manufactured in accordance with current European Union Good Manufacturing Practice.

Obicetrapib tablets are round, white film-coated tablets, with no identifying markings, containing 5 mg of obicetrapib calcium drug substance. The excipients present in the tablet cores are microcrystalline cellulose, mannitol, sodium starch glycollate, colloidal silicon dioxide, and magnesium stearate. A commercially available film-coating formula (Opadry II white, ex Colorcon) is applied to the cores.

Placebo tablets for obicetrapib are matching round, white film-coated tablets, with no identifying markings. The excipients present in the tablet cores are microcrystalline cellulose, mannitol, sodium starch glycollate, colloidal silicon dioxide, and magnesium stearate. A commercially available film-coating formula (Opadry II white, ex Colorcon) is applied to the cores.

Ezetimibe capsules are 10 mg ezetimibe tablets filled into capsule shells, 1 tablet per capsule. Each capsule also contains an excipient material, common to the tablets, as a filler to prevent the tablet from rattling in the capsule shell. Placebo capsules to match the ezetimibe capsules are the identical capsule shells filled with the excipient filler material only (no tablets).

Obicetrapib and placebo tablets and ezetimibe and placebo capsules will be packaged into foil blisters and assembled into blister cards providing the 2 study drugs for each treatment group. The blister cards will be clearly labelled to indicate which blisters to use on each day. Blister cards will be assembled into kits, and each kit will provide a sufficient supply for 1 month of dosing. The shelf-life will be assigned based on the stability of the individual products and will not be greater than the expiry date of the input ezetimibe tablets. The kits should be stored below 25° C.

Participants will be randomized to receive 2 of the study drugs listed above for the duration of the study. Both of the assigned study drugs will be administered by the participant orally and once daily on Days 1 through 57.

### Dosing Rationale

In previous multiple-dose clinical studies of obicetrapib in healthy subjects and patients, near maximal effects were observed with the 5 mg obicetrapib dose. At this dose level, CETP activity and concentrations were effectively reduced, and HDL-C levels were increased while LDL-C levels decreased. According to the Summary of Product Characteristics, the recommended daily dose of ezetimibe is 10 mg. Therefore, the present study will utilize a dose of 5 mg obicetrapib, administered with or without 10 mg ezetimibe, in participants with mild dyslipidaemia following the TLC diet.

### Administration Regime

Study drugs will be administered by the participant orally and once daily on Days 1 through 57. Study drugs should be administered at approximately the same time each morning, with food. On days with visits scheduled, study drugs should be administered with food following all fasted blood samples. If a participant forgets to take study drug on a given day, they should take the next dose as normal and should not take a double dose to make up for the forgotten dose.

### Study Design

This study is a placebo-controlled, double-blind, randomized Phase 2 study in participants with mild dyslipidaemia to evaluate the efficacy, safety, and tolerability of obicetrapib and ezetimibe combination therapy.

Participants, Investigators, the Clinical Research Organization, and the Sponsor will be blinded to all lipid results from Day 1 (Visit 2) for the first participant through the PK Visit (Visit 6) for the last participant in order to protect blinding to treatment assignment.

### Screening Period

At the Screening Visit (Visit 1), participants will be required to sign an informed consent form (ICF) before any study-related procedures are performed. After signing the ICF, participants will be assessed for study eligibility. Participants will also receive counselling regarding the Therapeutic Lifestyle Changes (TLC) diet and will receive instructions on how to follow this diet. The TLC diet was adapted from the United States National Institutes of Health Therapeutic Lifestyle Changes [TLC] diet and is illustrated below in Table 1. Participants will be required to begin the diet starting at the Screening Visit.

TABLE 1

| Component | Recommendation | |
|---|---|---|
| Saturated fats | <7% of total calories | |
| Total fat | 25% to 35% of total calories | |
| Dietary cholesterol | <200 | mg/day |
| Sodium | <2400 | mg/day |

| | |
|---|---|
| Total calories | Adjust total caloric intake to maintain desirable |
| | body weight and/or prevent weight gain |

If participants are screened on a day other than Day −1, then participants will return to the site for pharmacokinetic (PK) and CETP mass assessments on Day −1.

### Treatment Period

Up to 2 weeks after the Screening Visit, participants will return to the site on Day 1 (Visit 2) to be randomized and begin treatment. Approximately 100 eligible participants (25 participants per treatment group) will be randomized in a 1:1:1:1 ratio to 1 of the following treatment groups:
Combination therapy: 5 mg obicetrapib+10 mg ezetimibe;
Obicetrapib monotherapy: 5 mg obicetrapib+placebo ezetimibe;
Ezetimibe monotherapy: placebo obicetrapib+10 mg ezetimibe; or
Placebo: placebo obicetrapib+placebo ezetimibe.

During the 12-week Treatment Period, the assigned study drugs will be administered by the participant orally and once daily on Days 1 through 57. Participants will return to the site every 4 weeks for efficacy, safety, pharmacokinetic (PK), and CETP mass assessments. Participants, Investigators, the Clinical Research Organization, and the Sponsor will be blinded to all lipid results from Day 1 (Visit 2) for the first participant through the PK Visit (Visit 6) for the last participant in order to protect blinding to treatment assignment.

### Follow-up Period

Participants will return to the site for a Safety Follow-up Visit (Visit 6) approximately 4 weeks after the end of the Treatment Period for safety, PK, and CETP mass assessments.

### Pharmacokinetic Period

Participants will return to the site for a PK Visit (Visit 7) approximately 8 weeks after the end of the Treatment Period for PK and CETP mass assessments.

### Endpoints

### Efficacy Variables

The primary efficacy endpoint is percent change from Day 1 to Day 57 in LDL-C for the combination therapy group compared to the placebo group.

The secondary efficacy endpoints include the following, in hierarchical order:
Percent change from Day 1 to Day 57 in LDL-C for the obicetrapib monotherapy group compared to the placebo group;
Percent change from Day 1 to Day 57 in ApoB for the combination therapy group compared to the placebo group;
Percent change from Day 1 to Day 57 in ApoB for the obicetrapib monotherapy group compared to the placebo group;
Percent change from Day 1 to Day 57 in LDL-C for the combination therapy group compared to the ezetimibe monotherapy group;
Percent change from Day 1 to Day 57 in LDL-C for the obicetrapib monotherapy group compared to the ezetimibe monotherapy group;
Percent change from Day 1 to Day 57 in LDL-C for the ezetimibe monotherapy group compared to the placebo group; and
Percent change from Day 1 to Day 57 in ApoB for the combination therapy group compared to the ezetimibe monotherapy group.

The exploratory efficacy endpoints include the following:
Percent change from Day 1 to Day 57 in ApoB for the ezetimibe monotherapy group compared to the placebo group;
Pairwise comparisons among the various treatment groups for the percent change from Day 1 to Day 57 in non-HDL-C, VLDL-C, HDL-C, TG, ApoE and HDL-ApoE (with and without ApoC3);
Pairwise comparisons among the various treatment groups for the proportion of participants at Day 57 that achieve LDL-C<2.6 mmol/L (<100 mg/dL), LDL-C<1.8 mmol/L (<70 mg/dL), and LDL-C<1.3 mmol/L (<50 mg/dL);
Pairwise comparisons among the various treatment groups for the change from Day 1 to Day 57, Day 1 to Week 12, and Day 1 to Week 16 in the mean trough plasma levels of obicetrapib; and
Pairwise comparisons among the various treatment groups for the change from Day 1 to Day 57, Day 1 to Week 12, and Day 1 to Week 16 in CETP mass.

### Safety Variables

The safety and tolerability profiles of obicetrapib alone, ezetimibe alone, and obicetrapib in combination with ezetimibe will be assessed by clinical laboratory values and the incidence of (S)AEs.

### Statistical Analyses

### Analysis Populations

The Intent-to-Treat (ITT) Population will include all participants randomized into the study. Treatment classification will be based on the randomized treatment.

The Modified ITT (mITT) Population will include all participants in the ITT Population who receive at least 1 dose of any study drug and have a baseline value for the LDL-C assessment. Any efficacy measurement obtained after a participant receives a restricted lipid-altering therapy, outside of the current study design, will be removed from the mITT analysis. Treatment classification will be based on the randomized treatment. The mITT Population will be used for the primary analysis of all efficacy endpoints.

The Per-Protocol (PP) Population will include all participants in the mITT Population who have a baseline value for the LDL-C assessment, have a Day 85 value for the LDL-C assessment, and who did not experience a major protocol deviation that potentially impacted the primary efficacy endpoint. The PP Population, along with the reason for exclusion, will be finalized prior to study unblinding.

The PK Population will include all participants in the mITT Population who have sufficient blood samples collected for valid estimation of PK parameters.

The Safety Population will include all participants who receive at least 1 dose of any study drug. Treatment classification will be based on the actual treatment received. The Safety

Population will be the primary population used for the safety analyses.

### Statistical Methods

All study-collected data will be summarized by treatment group using descriptive statistics, graphs, and/or raw data listings. Descriptive statistics for continuous variables will include number of participants (n), mean, standard deviation (SD), median, minimum, and maximum values. Analysis of categorical variables will include frequency and percentage.

### Analysis of Efficacy

The mITT Population will be the primary population for the efficacy analyses. Efficacy will also be analysed using the ITT Population and the PP Population as supportive analyses.

### Primary Efficacy Analysis

performed using a mixed model for repeated measures (MMRM) approach. The analysis will include fixed effects for treatment, visit, and treatment-by-visit interaction, along with a covariate of the baseline value. The Restricted Maximum Likelihood estimation approach will be used with an unstructured covariance matrix. The least squares means, standard errors, and 2-sided 95% confidence intervals for each treatment group, for the pairwise comparisons of the combination therapy, obicetrapib monotherapy, and ezetimibe monotherapy to placebo, and for the pairwise comparison of combination therapy versus obicetrapib monotherapy will be provided. The treatment comparison will be performed using a 2-sided test at the α=0.05 level of significance.

Missing data will be imputed using multiple imputation methodology. Results will be combined using Rubin's method. Full details of the model and imputation will be provided in the Statistical Analysis Plan (SAP).

### Secondary and Exploratory Efficacy Analysis

A similar MMRM model will be used for the analysis of the secondary and exploratory efficacy endpoints corresponding to the percent change from baseline. For the binary efficacy endpoints, a logistic regression analysis will be performed with model covariates of treatment group and baseline LDL-C.

In order to maintain the overall Type I error rate, the secondary efficacy endpoints will be tested sequentially at the 0.05 significance level according to the pre-specified order of hierarchy. No adjustment will be made for multiplicity in testing the exploratory efficacy endpoints. Nominal p-values will be provided when applicable. Descriptive and graphical summaries by treatment group will also be presented. Any additional sensitivity and/or supplemental analyses will be defined in the SAP.

### Safety Analysis

The Safety Population will be the primary population for the safety analyses. All safety endpoints will be summarized descriptively. No statistical inference will be applied to the safety endpoints.

AEs will be categorized by primary system organ class and preferred term as coded using the Medical Dictionary for Regulatory Activities (MedDRA) category designations. Summaries of AEs, including the number and percentage of participants who experience an AE, will be provided.

Laboratory values will be summarized descriptively, including the change from baseline, by treatment group, and overall. In addition, shift tables will be presented to describe the change in laboratory parameter values at post-baseline visits using normal range categories (low, normal, and high).

### Pharmacokinetic Analysis

Plasma obicetrapib concentrations will be summarized with descriptive statistics based on the PK Population. Exploration of any relationships with obicetrapib exposure will be performed, as appropriate.

### Example 2b

Placebo-Controlled, Double-Blind, Randomized Phase 2 Study to Evaluate the Effect of Obicetrapib in Combination with Ezetimibe in Participants with Mild Dyslipidaemia and Potential Statin Intolerance (Analysis, Results & Conclusions)

### Participants, Treatment and Analysis

### Duration of Treatment:

8 weeks. Note: 44 participants completed the study under Clinical Study Protocol version 3.0 and received 12 weeks of treatment.

### Number of Participants:

Planned: 100 participants
Screened: 234 participants
Randomized: 112 participants (1 participant was randomized in error)
Completed treatment: 103 participants
Discontinued study: 14 participants
Discontinued treatment: 8 participants

### Diagnosis and Main Criteria for Inclusion:

Men or women 18 to 70 years of age, inclusive, with a BMI<40 kg/m2, fasting LDL-C levels>2.5 mmol/L (>100 mg/dL) and <4.5 mmol/L (<175 mg/dL) and TG levels<4.5 mmol/L (<400 mg/dL) were eligible to participate in the study. Participants were excluded from the study if they were currently taking any lipid-altering therapy, had any clinical manifestations of atherosclerotic cardiovascular disease or any evidence of ischemic coronary disease present on the 12-lead electrocardiogram (ECG) at the Screening Visit, had a diagnosis of type 1 or type 2 diabetes mellitus or glycosylated hemoglobin≥6.5% at the Screening Visit if no prior diagnosis of diabetes mellitus, or had uncontrolled hypertension, ie, sitting systolic blood pressure >160 mmHg and/or sitting diastolic blood pressure >90 mmHg taken as the average of triplicate measurements.

### Investigational Product and Comparator Information:

The study drugs consisted of 5 mg obicetrapib tablets or matching placebo tablets, and over-encapsulated 10 mg ezetimibe tablets or matching placebo capsules. All products were manufactured in accordance with the International Council for Harmonisation (ICH) current Good Manufacturing Practice.

### Statistical Methods:

### Analysis Populations:

The Intent-to-Treat (ITT) Population included all participants randomized into the study. Treatment classification was based on the randomized treatment.

The Modified Intent-to-Treat (mITT) Population included all participants in the ITT Population who received at least one dose of any study drug and had a baseline value for the LDL-C assessment. Any efficacy measurement obtained after a participant received a restricted lipid-altering therapy (any lipid-altering therapy other than the investigational study drugs were prohibited during the study), outside of the current study design, was removed from the mITT analysis. Treatment classification was based on the randomized treatment. The mITT Population was used for the primary analysis of all efficacy endpoints.

The Per-Protocol (PP) Population included all participants in the mITT Population who had a baseline value for the LDL-C assessment, had a Day 57 value for the LDL-C assessment, and did not experience a major Clinical Study Protocol deviation that potentially impacted the primary efficacy endpoint. Major Clinical Study Protocol deviations were defined in the Protocol Deviation Plan within the trial master file to align with ICH guidelines. The determination of membership in the PP Population was made prior to study unblinding. The PP Population was a secondary population for analysis of the primary efficacy endpoint.

The Safety Population included all participants who received at least 1 dose of any study drug. Treatment classification was based on the actual treatment received. The Safety Population was the primary population used for the safety analyses.

The PK Population included all participants in the mITT Population who had sufficient blood samples collected for valid estimation of PK parameters.

Counts and percentages of participants in each analysis population are summarized by treatment and in total based on all randomized participants. Reasons for exclusion from the PP Population are also summarized.

### Efficacy Analyses

The mITT Population was the primary population for the efficacy analyses. Efficacy was also analyzed using the ITT Population and the PP Population as supportive analyses for select endpoints.

LDL-C level was collected using the following 2 approaches:
1. At each scheduled visit, LDL-C level was calculated using the Friedewald formula; and
2. In addition, at baseline (Day 1) and at the end of the 8-week Treatment Period (Day 57), LDL-C was measured for all participants by preparative ultracentrifugation (PUC).
   Primary Efficacy Endpoint Primary analysis

The primary efficacy analysis of the percent change from Day 1 to Day 57 in LDL-C was performed using a mixed model for repeated measures (MMRM) approach. The analysis included fixed effects for treatment, visit, and treatment-by-visit interaction, along with a covariate of the baseline value as a continuous covariate. Randomization was stratified by categories of LDL-C value (<3.5 mmol/L [<135 mg/dL] or >3.5 mmol/L [≥135 mg/dL]) only to ensure similar distribution of LDL-C across all treatment groups; however, the MMRM model included the original scale of the LDL-C value as a continuous covariate. The Restricted Maximum Likelihood estimation approach was used with an unstructured covariance matrix. The least squares (LS) means, standard errors, and 2-sided 95% confidence intervals (CIs) for each treatment group, for the pairwise comparisons of the combination therapy, obicetrapib monotherapy, and ezetimibe monotherapy to placebo, and for the pairwise comparisons of combination therapy and obicetrapib monotherapy versus ezetimibe monotherapy are provided. The treatment comparison was performed using a 2-sided test at the α=0.05 level of significance.

The MMRM approach included all available assessments of percent change in LDL-C from baseline to Days 29, 57, and 85. The model assumed the data are missing at random. If any data were missing, the model used all information from the other time points to estimate the mean treatment difference at the given time point. No imputation of missing data was performed for the primary efficacy endpoint analysis.

### Primary Efficacy Endpoint Sensitivity Analysis

Sensitivity analyses were performed for the primary efficacy endpoint. In the first analysis, missing data were imputed using a control-based pattern imputation model assuming the data were missing not at random. The multiple imputation was performed such that only observations from the placebo group were used to derive the imputation model for missing

LDL-C values. Missing data at Days 29, 57, and 85 were imputed using multiple imputation methodology in 2 steps. Initially, 25 data sets were imputed for non-monotone missing values in the original dataset. In the second step, the remaining monotone missing values were imputed. Upon completion of the study, if the percentage of cases with incomplete data was larger than initially anticipated, then the number of imputations were increased for the final analysis.

The variables for the imputation model consisted of LDL-C values from baseline and Days 29, 57, and 85. For each imputation dataset, the percent change from baseline to Day 57 was analyzed using the MMRM model. The results from these 25 analyses were combined to construct the treatment estimates using the parameter estimates and associated standard errors. Similarly, the difference of the adjusted treatment means (combination therapy—placebo) is presented with the associated standard error and 95% CIs. Randomly chosen seed numbers were selected for the analysis and were retained.

In the second sensitivity analysis, the percent change from Day 1 to Day 57 in LDL-C, via the Friedewald formula, for the combination therapy group compared to the placebo group was analyzed using an Analysis of Covariance (ANCOVA) model with a fixed effect of treatment group and the baseline LDL-C value as a continuous covariate. The LS means, standard errors, and 2-sided 95% CIs for each treatment group, for the pairwise comparisons of the combination therapy, obicetrapib monotherapy, and ezetimibe monotherapy to placebo, are provided. The treatment comparison was performed using a 2-sided test at the α=0.05 level of significance. No imputation of missing data was performed for this sensitivity analysis.

In the third sensitivity analysis, the percent change from Day 1 to Day 57 in LDL-C was assessed using PUC, also referred to as beta quantification, to measure LDL-C levels. The percent change from Day 1 to Day 57 in LDL-C by PUC for the combination therapy group compared to the placebo group was analyzed using an ANCOVA similar to the model described in the second sensitivity analysis. No imputation of missing data was performed for this sensitivity analysis.

In the fourth sensitivity analysis, a similar ANCOVA model as described above was utilized for the assessment of percent change from baseline to Day 57 in LDL-C as measured by PUC. In this analysis, missing data at Day 57 were imputed using a last observation carried backward (LOCB) approach in which the next available LDL-C measurement, as measured by PUC, following Day 57 was used in the analysis. The previous TULIP trial (NCT01970215) observed similar values of percent change in LDL-C at Weeks 8 and 12, providing a rationale for the LOCB approach in this study.

### Secondary Efficacy Endpoints

Similar MMRM models were developed to examine the percent change from Day 1 to Day 57 in LDL-C (for other pairwise treatment group comparisons beside the analysis specified for the primary efficacy endpoint, using the Friedewald formula) or ApoB. In order to maintain the overall Type 1 error rate, the secondary efficacy endpoints were tested sequentially at the 0.05 significance level according to the pre-specified order of hierarchy. This applied to the following analyses:
Percent change from Day 1 to Day 57 in LDL-C for the obicetrapib monotherapy group compared to the placebo group;
Percent change from Day 1 to Day 57 in ApoB for the combination therapy group compared to the placebo group;
Percent change from Day 1 to Day 57 in ApoB for the obicetrapib monotherapy group compared to the placebo group;
Percent change from Day 1 to Day 57 in LDL-C for combination therapy group compared to the ezetimibe monotherapy group;
Percent change from Day 1 to Day 57 in LDL-C for the obicetrapib monotherapy group compared to the ezetimibe monotherapy group;
Percent change from Day 1 to Day 57 in LDL-C, for the ezetimibe monotherapy group compared to the placebo group; and
Percent change from Day 1 to Day 57 in ApoB for the combination therapy group compared to the ezetimibe monotherapy group.

### Exploratory Efficacy Endpoints

Similar MMRM models were used for the exploratory efficacy endpoints. The proportion of participants at Day 57 that achieved LDL-C<2.6 mmol/L (<100 mg/dL), LDL-C<1.8 mmol/L (<70 mg/dL), and LDL-C<1.3 mmol/L (<50 mg/dL) were examined through Logistic Regression Models for binary variables with covariates of the treatment groups and the baseline variable.

No adjustment was made for multiplicity in testing the exploratory efficacy endpoints. Nominal p-values are provided when applicable.

### Safety Analyses:

The Safety Population was the primary population for the safety analyses. All safety endpoints are summarized descriptively. No statistical inference was applied to the safety endpoints.

AEs were categorized by primary system organ class (SOC) and preferred term (PT) as coded using the Medical Dictionary for Regulatory Activities (version 23.0) category designations. Summaries of AEs, including the count and percentage of participants who experienced an AE, are provided.

An overview of treatment-emergent adverse events (TEAEs) is provided, including counts and percentages of participants (and event counts).

Listings are presented specifically for TEAEs, treatment-emergent serious adverse events (TESAEs), and TEAEs leading to discontinuation of study drug.

For clinical laboratory tests, laboratory values are summarized descriptively, including the change from baseline, by treatment, and in total. In addition, shift tables are presented to describe the change in laboratory parameter values at post-baseline visits using normal range categories (low, normal, and high).

For vital signs, values and changes from baseline are summarized with descriptive statistics at each visit by treatment.

Summary statistics for 12-lead ECG data are provided for continuous results (PR, QRS, heart rate, RR, QT, QTc, and QTcF) and the overall interpretation by treatment and in total.

### Efficacy Results

This study was a placebo-controlled, double-blind, randomized Phase 2 study in participants with mild dyslipidemia to evaluate the efficacy, safety, and tolerability of obicetrapib and ezetimibe combination therapy. The primary efficacy endpoint was percent change from Day 1 to Day 57 in LDL-C for the obicetrapib 5 mg+ezetimibe 10 mg group compared to the placebo group.

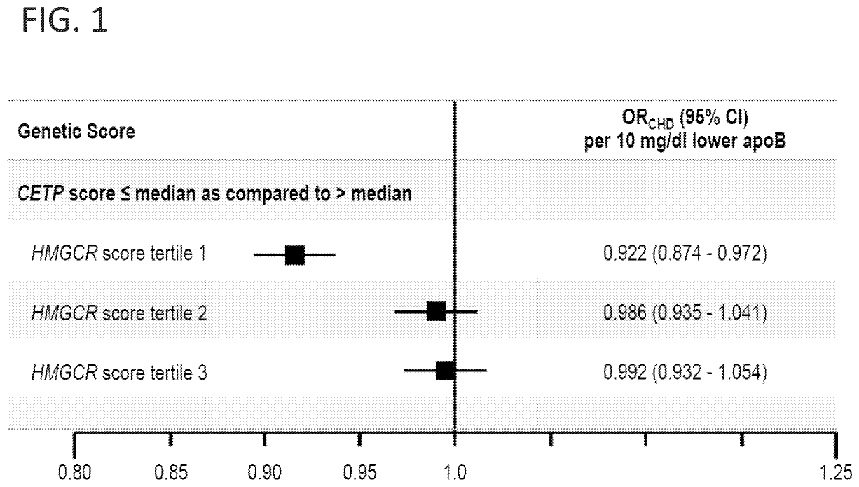

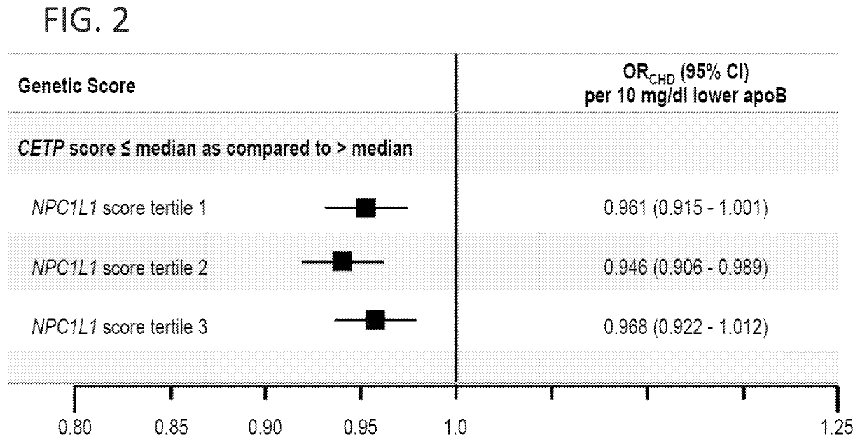

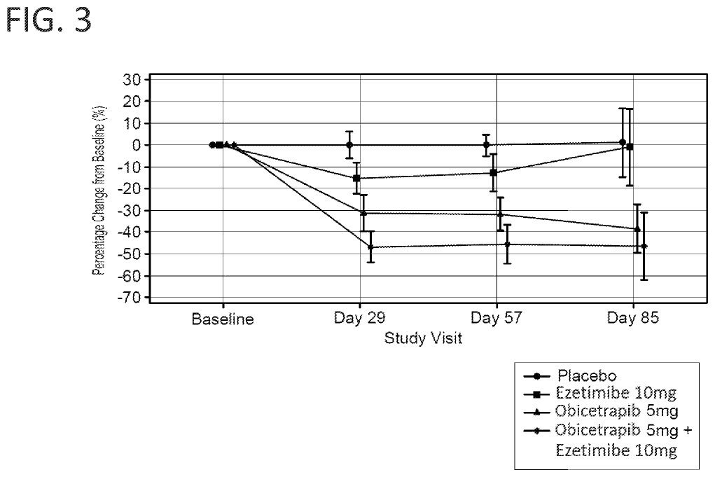

At Day 57, the mean LDL-C by Friedewald formula was 71.3 mg/dL for the obicetrapib 5 mg+ezetimibe 10 mg group, 87.0 mg/dL for the obicetrapib 5 mg group, 109.1 mg/dL for the ezetimibe 10 mg group, and 136.2 mg/dL for the placebo group. The mean percent change from baseline to Day 57 in LDL-C by Friedewald formula was −45.63% for the obicetrapib 5 mg+ezetimibe 10 mg group, −31.88% for the obicetrapib 5 mg group, −12.69% for the ezetimibe 10 mg group, and −0.17% for the placebo group. In this regard, reference is made to Table 2 and FIG. 3.

The PUC and Martin/Hopkins calculation LDL-C values were similar to the Friedewald formula values for Day 57.

At Day 57, both the obicetrapib 5 mg group and the obicetrapib 5 mg+ezetimibe 10 mg group demonstrated significant reductions in the percent change from baseline, compared to placebo, for ApoB and non-HDL-C. In this regard, reference is made to Table 3.

At Day 57, both the obicetrapib 5 mg group and the obicetrapib 5 mg+ezetimibe 10 mg group demonstrated significant increases in the percent change from baseline, compared to placebo, for HDL-C and ApoE.

The number of participants that achieved an LDL-C<70 mg/dL by Friedewald formula at Day 57 was 13 (48.1%) participants in the obicetrapib 5 mg+ezetimibe 10 mg group, 7 (25.0%) participants in the obicetrapib 5 mg group, 1 (3.6%) participant in the ezetimibe 10 mg group, and 0 (0.0%) participants in the placebo group. The number of participants that achieved an LDL-C<100 mg/dL by Friedewald formula at Day 57 was 21 (77.8%) participants in the obicetrapib 5 mg+ezetimibe 10 mg group, 16 (57.1%) participants in the obicetrapib 5 mg group, 8 (28.6%) participants in the ezetimibe 10 mg group, and 2 (7.1%) participants in the placebo group.

In conclusion, this study met its primary efficacy objective. The combination of obicetrapib 5 mg+ezetimibe 10 mg markedly reduced LDL-C, non-HDL-C, and ApoB; increased HDL-C and ApoE; and increased the proportion of participants who achieved LDL-C below clinically-relevant thresholds of 100 mg/dL and 70 mg/dL, in comparison to both placebo and ezetimibe 10 mg monotherapy.

### Safety Results

The majority of participants in the placebo, ezetimibe 10 mg, and obicetrapib 5 mg treatment groups were exposed to the study drug for 8 to <12 weeks.

Overall, 27 (24.3%) participants experienced at least 1 TEAE: 9 (33.3%) participants in the obicetrapib 5 mg+ezetimibe 10 mg group, 4 (14.3%) participants in the obicetrapib 5 mg group, 8 (28.6%) participants in the ezetimibe 10 mg group, and 6 (21.4%) participants in the placebo group.

The majority of the TEAEs were mild or moderate in severity. In total, 9 (8.1%) participants experienced a TEAE with a maximum severity of moderate: 2 (7.4%) participants in the obicetrapib 5 mg+ezetimibe 10 mg group, 1 (3.6%) participant in the obicetrapib 5 mg group, 4 (14.3%) participants in the ezetimibe 10 mg group, and 2 (7.1%) participants in the placebo group. One (0.9%) participant (Participant 106-010) in the ezetimibe 10 mg group experienced a severe TEAE of multiple fractures.

The most common SOCs of TEAEs were gastrointestinal disorders (9 [8.1%] participants), infections and infestations (6 [5.4%] participants), and nervous system disorders (6 [5.4%] participants). The most common PTs experienced among all participants were diarrhea and headache (4 [3.6%] participants each).

Overall, 11 (9.9%) participants experienced at least 1 study drug-related TEAE: 3 (11.1%) participants in the obicetrapib 5 mg+ezetimibe 10 mg group, 1 (3.6%) participant in the obicetrapib 5 mg group, 3 (10.7%) participants in the ezetimibe 10 mg group, and 4 (14.3%) participants in the placebo group. The most common SOC of study drug-related TEAEs was gastrointestinal disorders (7 [6.3%] participants).

In total, there were 4 (3.6%) participants who experienced at least 1 TEAE that led to discontinuation of the study drug: 2 (7.4%) participants in the obicetrapib 5 mg+ezetimibe 10 mg group and 1 (3.6%) participant each in the ezetimibe 10 mg group and the placebo group. Two (1.8%) participants overall, both in the ezetimibe 10 mg group, experienced a TESAE.

There were no deaths in this study.

Two participants in the ezetimibe 10 mg group experienced serious adverse events (SAEs). Participant 004-003 experienced an SAE of hyperventilation that was rated as moderate and not related to the study drug. Participant 106-010 experienced an SAE of multiple fractures that was rated as severe and not related to the study drug. Both participants recovered from these SAEs.

There were no clinically significant chemistry, hematology, or urinalysis parameters.

In general, no clinically meaningful changes in vital signs, physical examinations, or ECGs were observed. The differences in the mean blood pressure values did not show any notable increases or trends across treatment groups.

Consistent with previous studies, treatment with obicetrapib 5 mg, both as monotherapy and in combination with ezetimibe 10 mg, was generally safe and well tolerated.

### CONCLUSIONS

This was a placebo-controlled, double-blind, randomized Phase 2 study to evaluate the effect of obicetrapib in combination with ezetimibe in participants with mild dyslipidemia.

The key findings are as follows:
The combination therapy of obicetrapib 5 mg+ezetimibe 10 mg markedly reduced LDL-C, non-HDL-C, and ApoB, and increased HDL-C and ApoE;
The combination therapy of obicetrapib 5 mg+ezetimibe 10 mg significantly increased the proportion of participants who achieved LDL-C below clinically-relevant thresholds of 100 mg/dL and 70 mg/dL, in comparison to both placebo and ezetimibe 10 mg monotherapy;
The combination therapy of obicetrapib 5 mg+ezetimibe 10 mg notably reduced LDL-C, non-HDL-C, and ApoB compared with obicetrapib 5 mg monotherapy;
The PUC and Martin/Hopkins calculations of LDL-C values were similar to the Friedewald formula values for Day 57 in this population;
The PK profile for obicetrapib was consistent with previous findings and showed almost complete elimination of obicetrapib by 8 weeks after long-term daily dosing; and
The study drug was well tolerated, with no evidence of any safety signals related to obicetrapib therapy.

In conclusion, treatment with obicetrapib 5 mg alone or in combination with ezetimibe 10 mg was well tolerated and resulted in a significant improvement in lipid profiles in participants with mild dyslipidemia, with a shift toward a less atherogenic lipid profile.

TABLE 2
Summary of LDL-C (mg/dL) by Friedewald Formula - mITT Population

| | | | | Obicetrapib 5 mg + |
|---|---|---|---|---|
| Visit | Placebo | Ezetimibe 10 mg | Obicetrapib 5 mg | Ezetimibe 10 mg |
| Statistic | (N = 28) | (N = 28) | (N = 28) | (N = 27) |

Baseline

| n | 28 | | 28 | | 28 | | 27 | |
|---|---|---|---|---|---|---|---|---|
| Mean (SD) | 136.8 | (19.86) | 128.0 | (22.33) | 127.5 | (20.87) | 132.2 | (27.48) |

| | | | | |
|---|---|---|---|---|
| Median | 137.5 | 129.0 | 126.0 | 131.0 |

Day 57

| n | 27 | | 25 | | 25 | | 24 | |
|---|---|---|---|---|---|---|---|---|
| Mean (SD) | 136.2 | (25.88) | 109.1 | (21.15) | 87.0 | (28.61) | 71.3 | (27.44) |

| | | | | |
|---|---|---|---|---|
| Median | 138.0 | 109.0 | 87.0 | 65.5 |

Percent change from baseline to Day 57 (%)

| n [symbol] | 27 | | 25 | | 25 | | 24 | |
|---|---|---|---|---|---|---|---|---|
| Mean (SD) | −0.17 | (12.682) | −12.69 | (21.316) | −31.88 | (18.860) | −45.63 | (21.677) |

| | | | | |
|---|---|---|---|---|
| Median | −1.40 | −14.80 | −34.40 | −51.95 |

Change from baseline to Day 57

| n [symbol] | 27 | | 25 | | 25 | | 24 | |
|---|---|---|---|---|---|---|---|---|
| Mean (SD) | −0.3 | (17.34) | −18.0 | (26.01) | −39.5 | (21.54) | −62.5 | (34.46) |

| | | | | |
|---|---|---|---|---|
| Median | −2.0 | −19.0 | −41.0 | −60.5 |

Baseline was defined as the last measurement prior to the first dose of study drug.
n [symbol] = number of participants with a measurement at both baseline and the specific scheduled visit.
LDL-C = low-density lipoprotein cholesterol; mITT = Modified Intent-to-Treat; SD = standard deviation. [symbol]
indicates data missing or illegible when filed

TABLE 3
Summary of ApoB (mg/dL) - mITT Population

| | | | | Obicetrapib 5 mg + |
|---|---|---|---|---|
| Visit | Placebo | Ezetimibe 10 mg | Obicetrapib 5 mg | Ezetimibe 10 mg |
| Statistic | (N = 28) | (N = 28) | (N = 28) | (N = 27) |

Baseline

| n | 28 | | 28 | | 28 | | 27 | |
|---|---|---|---|---|---|---|---|---|
| Mean (SD) | 108.0 | (14.92) | 104.4 | (14.57) | 101.0 | (15.12) | 106.1 | (19.83) |

| | | | | |
|---|---|---|---|---|
| Median | 105.5 | 103.0 | 102.0 | 105.0 |

Day 57

| n | 27 | | 25 | | 26 | | 24 | |
|---|---|---|---|---|---|---|---|---|
| Mean (SD) | 106.9 | (19.42) | 94.8 | (17.67) | 78.3 | (16.19) | 71.5 | (14.87) |

| | | | | |
|---|---|---|---|---|
| Median | 107.0 | 94.0 | 75.0 | 73.0 |

Percent change from baseline to Day 57 (%)

| n [symbol] | 27 | | 25 | | 26 | | 24 | |
|---|---|---|---|---|---|---|---|---|
| Mean (SD) | −1.17 | (11.692) | −8.78 | (14.445) | −22.15 | (13.356) | −31.62 | (15.521) |

| | | | | |
|---|---|---|---|---|
| Median | −0.90 | −8.90 | −23.50 | −34.75 |

Change from baseline to Day 57

| n [symbol] | 27 | | 25 | | 26 | | 24 | |
|---|---|---|---|---|---|---|---|---|
| Mean (SD) | −1.3 | (12.90) | −9.5 | (15.76) | −22.6 | (13.63) | −34.8 | (19.06) |

| | | | | |
|---|---|---|---|---|
| Median | −1.0 | −8.0 | −24.0 | −34.0 |

Baseline was defined as the last measurement prior to the first dose of study drug.
n [symbol] = number of participants with a measurement at both baseline and the specific scheduled visit.
ApoB = apolipoprotein B; mITT = Modified Intent-to-Treat; SD = standard deviation. [symbol]
indicates data missing or illegible when filed

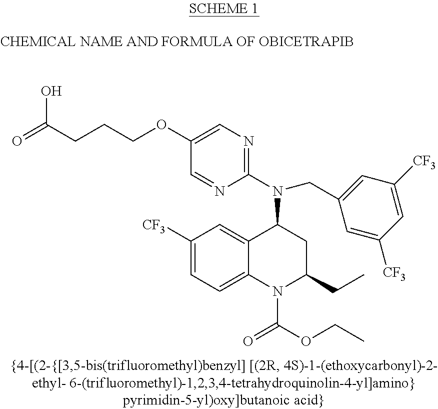 [SMILES: CCOC(=O)N1c2ccc(C(F)(F)F)cc2[C@@H](N(Cc2cc(C(F)(F)F)cc(C(F)(F)F)c2)c2ncc(OCCCC(=O)O)cn2)C[C@H]1CC]

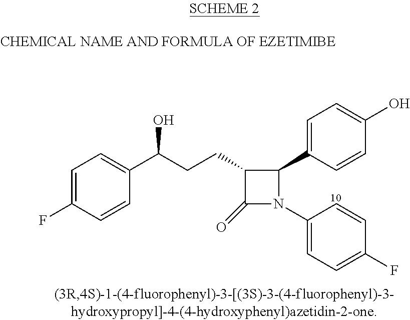 [SMILES: O=C1[C@H](CC[C@H](O)c2ccc(F)cc2)[C@@H](c2ccc(O)cc2)N1c1ccc(F)cc1]

1-34. (canceled)

35. A method of treating a subject suffering from or having an increased risk for hyperlipidemia or mixed dyslipidemia, the method comprising:
administering to the subject a therapeutically effective amount of (i) obicetrapib, or a pharmaceutically acceptable salt or solvate thereof, and (ii) ezetimibe, or a pharmaceutically acceptable salt or solvate thereof,
wherein the subject is partially or completely intolerant to statins.

36. The method according to claim 35, wherein the subject suffers from hyperlipidemia or mixed dyslipidemia.

37. The method according to claim 35, wherein the subject has an increased risk for hyperlipidemia or mixed dyslipidemia.

38. The method according to claim 35, wherein the subject is orally administered between 1 to 10 mg per day obicetrapib and between 5 to 20 mg ezetimibe per day.

39. The method according to claim 38, wherein the subject is orally administered (i) 5 mg obicetrapib per day and 10 mg ezetimibe per day or (ii) 10 mg obicetrapib per day and 10 mg ezetimibe per day.

40. The method according to claim 35, wherein apolipoprotein B (ApoB) concentration in the blood of the subject is reduced.

41. The method according to claim 35, wherein LDL-C concentration in the blood of the subject is reduced.

42. The method according to claim 35, wherein the reduction of LDL-C levels by the combination of obicetrapib and ezetimibe is no less than the LDL-C reduction by the same dose of obicetrapib administered as monotherapy.

43. The method according to claim 42, wherein the LDL-C reduction by a daily dose of 5 mg obicetrapib when administered with 10 mg ezetimibe is no less than the LDL-C reduction by a daily dose of 5 mg obicetrapib administered as monotherapy.

44. The method according to claim 42, wherein the relative LDL-C reduction by a daily dose of 10 mg obicetrapib when administered as the pharmaceutical composition with 10 mg ezetimibe is similar to the relative LDL-C reduction by a daily dose of 10 mg obicetrapib administered as monotherapy.

45. The method according to claim 35, wherein obicetrapib and ezetimibe are administered in a single oral dosage form.

46. The method according to claim 45, wherein the oral dosage form is an oral fixed dose combination.

47. The method according to claim 46, wherein the oral fixed dose combination is a solid dosage form.

48. The method according to claim 47, wherein the solid dosage form is a capsule or tablet.

49. The method according to claim 35, wherein the method reduces the risk for major adverse cardiovascular events.

50. A method of treating a subject suffering from cardiovascular disease or having an increased risk for cardiovascular disease, the method comprising:
administering to the subject a therapeutically effective amount of (i) a CETP-inhibitor, or a pharmaceutically acceptable salt or solvate thereof, and (ii) ezetimibe, or a pharmaceutically acceptable salt of solvate thereof,
wherein the subject is partially or completely statin intolerant.

51. The method of claim 50, wherein the CETP-inhibitor is obicetrapib.

52. A method for reducing the risk for cardiovascular events in a subject in need thereof, the method comprising:
administering to the subject a therapeutically effective amount of (i) obicetrapib, or a pharmaceutically acceptable salt or solvate thereof, and (ii) ezetimibe, or a pharmaceutically acceptable salt or solvate thereof,
wherein the subject is partially or completely statin intolerant.
